(19) United States
(12) Patent Application Publication
(10) Pub. No.: US 2006/0058742 A1
(43) Pub. Date: Mar. 16, 2006

(54) Protective cover for arterial needle

(76) Inventors: Sedo Cha, Medford, NJ (US); Allan F. Wills, Marlton, NJ (US)

Correspondence Address: Hollstein Keating Cattell Johnson & Goldstein P.C.;Willow Ridge Executive Office Park, Suite 301, 750 Route 73 South, Marlton, NJ, 08053, US

(21) Appl. No.: 10/942,705

(22) Filed: Sep. 15, 2004

(51) Int. Cl. A61M 5/00

(52) U.S. Cl. 604/263

(57) ABSTRACT

A protective cover for arterial or a similar needle is effective for covering the needle tip and also can be retracted along the needle shaft to uncover and reopen the needle. The cover has a sheath section, whose movement along the shaft is controlled by an actuator section. The sheath section has two through channels and the actuator section has an actuator element with a corresponding channel opening. In the open position, that is when the needle tip is exposed, the channels within the sheath section and the channel opening of the actuator element are aligned and the needle shaft extends through the channels and the opening. To enclose the needle tip, the cover is slid along the needle shaft to the tip, so that the actuator section is slid off the shaft. At this point, a spring within the actuator section compels the actuator element upward, effectively locking the tip within the cover. Pushing down on the actuator element, realigns to channels and channel opening and permits the cover to be slid back on the shaft and retracted off the tip, reopening the needle. A tether is provided to limit the movement of the cover along the needle shaft.

### BACKGROUND OF THE INVENTION

The need to ensure that medical needles, including arterial needles, are properly sheathed when not in use is of critical importance in the healthcare industry. It is necessary that provision is made to safely handle needles both as they are being used and afterward, during the disposal process. Of course, it is the extremely sharp tip of the needle which creates a puncture hazard; but if the needle has been contaminated and the skin is pricked or even touched, there is also a real risk of exposure to disease, especially when dealing with highly contagious diseases such as AIDS and hepatitis. This risk of contamination is especially present during the disposal of needles.

To address this hazardous situation, the medical appliance industry has developed protective covers or guards which are designed to be positioned over the sharp tips of needle shafts. Immediately after a needle is used, a protective cover is placed over the tip, before the health care professional handles the needle any further. When being disposed, the needle remains enclosed. The hazards presented by sharp, contaminated needles are drastically reduced.

There are many different protective covers or guards in the industry. See, for example, those disclosed in U.S. Pat. Nos. 4,929,241, 5,098,401, 5,344,408, 6,001,080, 6,210,373, and 6,623,458. However, although different, the various prior art protective devices have one thing in common; they are generally all designed for single usage applications. In other words, needles are to be used one time and once the covers discussed in the prior art are positioned over the needle tips, the covers are not normally intended to be removable so that the needles can be re-used. In most cases, the needles are then designed to be discarded after a single use.

This presents a glaring shortcoming, because often times it may be necessary or advantageous to withdraw and then reuse the needles. Nonetheless, safety dictates that needle tips be covered when not in use. Yet once a cover encloses a tip, it cannot or is not designed to be removed and the needle must be discarded—compelling the use of another needle. There is no effective needle safety cover that can be locked in position to enclose a needle tip, but then be removed for extended needle usage or to allow the needle to be autoclaved.

### SUMMARY OF THE INVENTION

It is thus the object of the present invention to overcome the disadvantages and shortcomings of prior arterial and similar needle protective safety covers.

It is the object of the present invention to provide a protective cover for an arterial needle which provides an efficient and effective enclosure of the needle tip.

It is another object of the present invention to provide a protective cover for an arterial needle which can be used to securely enclose the tip of the needle and then can be removed from the tip to reopen the needle and allow needle reuse.

It is a further object of the present invention to provide a protective cover for an arterial needle which is readily and easily retractable along the shaft of the needle.

It is still another object of the present invention to provide a protective cover for an arterial needle which allows for the safe and easy placement of the cover onto and off the needle tip.

It is a further object of the present invention to provide a protective cover for an arterial needle which, when positioned over the needle tip, ensures for a positive and secure locked enclosure of the tip.

It is still another object of the present invention to provide a protective cover for an arterial needle which permits the needle to be autoclaved.

It is another object of the present invention to provide a protective cove for an arterial needle which is relatively simple, but durable in design, and which can be manufactured economically.

It is a further object of the present invention to provide a protective cover for an arterial needle which is not restricted by the size of the needle, since it can be used on needles of any length and needle gauge.

These and other objects are accomplished by the present invention, a protective cover for arterial needles which is effective in covering the needle tip, but which also can be retracted along the needle shaft to uncover and reopen the needle. The cover has a sheath section, whose movement along the shaft is controlled by an actuator section. The sheath section has two through channels and the actuator section has an actuator element with a corresponding channel opening. In the open position, that is when the needle tip is exposed, the channels within the sheath section and the channel opening of the actuator element are aligned and the needle shaft extends through the channels and the opening. To enclose the needle tip, the cover is slid along the needle shaft to the tip, so that the actuator section is slid off the shaft. At this point, a spring within the actuator section compels the actuator element upward, effectively locking the tip within the cover. Pushing down on the actuator element realigns to channels and channel opening and permits the cover to be slid back on the shaft and retracted off the tip, reopening the needle. A tether is provided to limit the movement of the cover along the needle shaft.

Novel features which are considered as characteristic of the invention are set forth in particular in the dependent claims. The invention, itself, however, both as to its design, construction and use, together with the additional features and advantages thereof, are best understood upon review of the following detailed description with reference to the accompanying drawings.

### BRIEF DESCRIPTION OF THE DRAWINGS

FIG. 1 is an elevation, cross-sectional view of the cover of the present invention in use with an arterial needle.

FIG. 2 is an elevation view of the cover of the present invention as it is slid towards the tip of an arterial needle.

FIG. 3 is an elevation, cross-sectional view of the cover of the present invention in place enclosing the tip of an arterial needle.

FIG. 4 is an elevation, cross-sectional view of another embodiment of the cover of the present invention in use with an arterial needle.

FIG. 5 is an elevation view of the second embodiment of the cover of the present invention as it is slid towards the tip of an arterial needle.

FIG. 6 is an elevation, cross-sectional view of the second embodiment of the cover of the present invention in place enclosing the tip of an arterial needle.

### DETAILED DESCRIPTION OF THE INVENTION

Arterial needle 1 comprises base member 2 and needle shaft 4 with needle tip 6. Cover 10 is configured to be slideably mounted on shaft 4. Cover 10 has a sheath section 12 with channels 14 and 16 extending therethrough. Sheath section 12 further comprises outwardly extending actuator section 18 with lower body portion 22. Positioned within actuating section 18 is actuator element 24 which has channel opening 20. When cover 10 is positioned on shaft 4, channel opening 20 is aligned with channels 14 and 16. A biasing means, such as spring 26, is positioned within actuator section 18 and acts to provide an expansive upward force against the element.

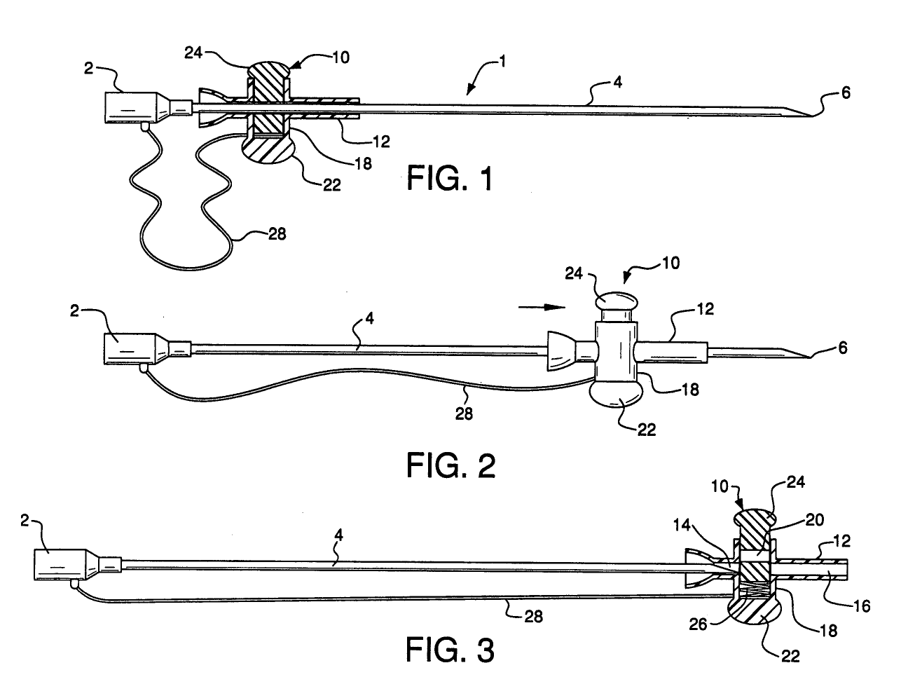

When needle 1 is being used, cover 10 is positioned adjacent to base member 2, as seen in FIG. 1. Needle shaft 4 extends through aligned channels 14 and 16 and channel opening 20, as described above. In this position, spring 26 is compressed between actuator element 24 and body portion 22 of actuator section 18. The presence of shaft 4 within channels 14 and 16 and channel opening 20, maintains cover 10 in position on the shaft.

When needle tip 6 is to be enclosed, cover 10 is slid along shaft 4 towards the tip, as shown in FIG. 2. When actuator section 18 is slid off shaft 4, the shaft is no longer in channel opening 20 and, as a result, it can no longer can act against the expansive force of spring 26. As a result, spring 26 expands, compelling actuator element 24 upward. This effectively encloses and locks sheath section 12 of cover 10, over needle tip 6, as shown in FIG. 3.

Tether strap 28 connects cover 10 with base member 2. Tether strap 28 is specifically sized to limit the movement of cover 10 along shaft 4 and to ensure that the cover can travel just far enough past tip 6, such that the shaft is slid out of channel opening 20, causing the action of spring 26 to lift actuator element 24 and lock cover 10 in place.

When needle 1 is to be immediately reused or prepared to be autoclaved, actuator element 24 is simply depressed. This results in spring 26 being compressed, again aligning channel opening 20 with channels 14 and 16 so that shaft 4 can reenter these passageways. Cover 10 can then be retractably slid back on shaft 4 towards base member 2.

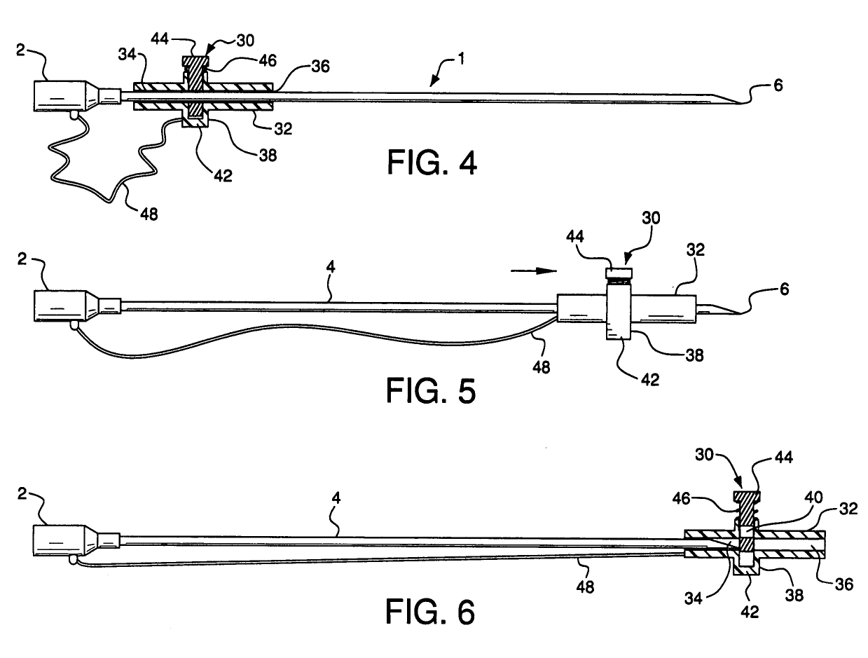

FIGS. 4-6 show an alternate configuration of the cover envisioned by the present invention. As with the embodiment previously described, cover 30 is configured to be slideably mounted on shaft 4. Cover 30 has a sheath section 32 with channels 34 and 36 extending therethrough. Sheath section 32 further comprises outwardly extending actuator section 38 with lower body portion 42. Positioned within actuating section 18 is actuator element 44 which has channel opening 40. When cover 30 is positioned on shaft 4, channel opening 40 is aligned with channels 34 and 36. A biasing means, such as spring 46, is positioned between actuator section 38 and actuator element 44 and acts to provide an expansive upward force against the element.

When needle 1 is being used, cover 30 is positioned adjacent to base member 2, as seen in FIG. 4. Needle shaft 4 extends through aligned channels 34 and 36 and channel opening 40, as described above. In this position, spring 46 is compressed between actuator element 44 and actuator section 38. The presence of shaft 4 within channels 34 and 36 and channel opening 40, maintains cover 30 in position on the shaft.

When needle tip 6 is to be enclosed, cover 30 is slid along shaft 4 towards the tip, as shown in FIG. 5. When actuator section 38 is slid off shaft 4, the shaft is no longer in channel opening 40 and, as a result, it can no longer can act against the expansive force of spring 46. As a result, spring 46 expands, compelling actuator element 44 upward. This effectively encloses and locks sheath section 32 of cover 40, over needle tip 6, as shown in FIG. 6.

Tether strap 48 connects cover 30 with base member 2. Tether strap 48 is specifically sized to limit the movement of cover 30 along shaft 4 and to ensure that the cover can travel just far enough past tip 6, such that the shaft is slid out of channel opening 40, causing the action of spring 46 to lift actuator element 44 and lock cover 30 in place.

When needle 1 is to be immediately reused or prepared to be autoclaved, actuator element 44 is simply depressed. This results in spring 46 being compressed, again aligning channel opening 40 with channels 34 and 36 so that shaft 4 can reenter these passageways. Cover 30 can then be retractably slid back on shaft 4 towards base member 2.

It can thus be seen that covers 10 and 30 provide permanently attached, user-friendly, yet effective safety enclosures for arterial and similar needles. They further permit the needles to be reopened for immediate reuse or to allow the needles to be autoclaved and used again. The covers themselves are shaped for convenient and efficient use, however it is contemplated that a variety of cover shapes and configurations can be employed without departing from the spirit and intent of the invention. The scope of the invention is not to be considered restricted by the shape of the cover.

Similarly, while a single tether 28 is shown, it is envisioned that two or more tethers may be advantageously employed, as necessary, to ensure that the cover is moved the requisite distance along the needle shaft.

Certain novel features and components of this invention are disclosed in detail in order to make the invention clear in at least one form thereof. However, it is to be clearly understood that the invention as disclosed is not necessarily limited to the exact form and details as disclosed, since it is apparent that various modifications and changes may be made without departing from the spirit of the invention.

1. A retractable protective cover for an arterial needle with a base member and a needle shaft with a tip extending therefrom, said cover comprising:
(a) sheath means slideably mounted on the needle shaft for forming a protective enclosure around the tip of the needle shaft, said sheath means comprising channel means for receiving the needle shaft and for allowing slideable movement of the cover along the needle shaft; and
(b) actuator means mounted in spaced relation the sheath means for controlling the sliding movement of the sheath means between the base member and the tip of the needle shaft and for locking the cover over the tip of the needle shaft, said actuator means comprising opening means which, when aligned with the channel means, allows the cover to slideably move along the needle shaft within the channel means and the opening means and when not aligned with the channel means, secures the cover in position over the tip of the needle shaft.

2. The protective cover as in claim 1 further comprises biasing means for providing an expansive force on the actuator means.

3. The protective cover as in claim 2 whereby the application of pressure on the actuator means in opposition to the expansive force of the biasing means when the cover is secured in position over the tip of the needle shaft, aligns the opening means with the channel means to allow the cover to be slideably moved along the needle shaft.

4. The protective cover as in claim 2 whereby upon application of pressure on the actuator means in opposition to the expansive force of the biasing means when the cover is secured in position over the tip of the needle shaft, the cover can be unlocked and retractably slid along the needle shaft towards the base member.

5. The protective cover as in claim 1 further comprising tethering means between the cover and the base member for limiting the movement of the cover in relation to the base member.

6. A retractable protective cover for an arterial needle with a base member and a needle shaft with a tip extending therefrom, said cover comprising:
(a) a sheath having first and second channels extending therethrough, said channels being configured for receiving the needle shaft and for allowing slideable movement of the cover along the needle shaft;
(b) an actuator section mounted in spaced relation to the sheath, said actuator section having an opening therethrough, whereby when the opening and first and second channels are aligned, the cover is slideable along the needle shaft;
(c) biasing means for providing a force within the actuator section whereby when the cover is located over the tip of the needle shaft and the opening and first and second channels are not aligned, the cover is located in position over the tip and, upon application of pressure on the actuator section in opposition to the expansive force of the biasing means when the cover is located over the tip, the opening and first and second channels are aligned and the cover is unlocked and can be retractably slid along the needle shaft towards the base member.

7. The protective cover as in claim 6 further comprising a tether connecting the cover to the base member.

8. The protective cover as in claim 6 wherein the sheath comprises two sections each section containing one of the two channels.

9. The protective cover as in claim 6 wherein the actuator section extends from the sheath.

10. The protective cover as in claim 6 wherein the biasing means comprises a spring.

11. The protective cover as in claim 6 wherein the actuator section comprises an actuator element.

12. The protective cover as in claim 9 wherein the actuator section further comprises an actuator element.
